(19) United States
(12) Patent Application Publication
(10) Pub. No.: US 2024/0411959 A1
(43) Pub. Date: Dec. 12, 2024

(54) PERFORMANCE EVALUATION APPARATUS FOR VIBRATION PROOF RUBBER

(71) Applicant: Honda Motor Co., Ltd., Tokyo (JP)

(72) Inventors: Nao SUGIMOTO, Saitama (JP); Takeshi KASAI, Tokyo (JP)

(73) Assignee: Honda Motor Co., Ltd., Tokyo (JP)

(21) Appl. No.: 18/655,328

(22) Filed: May 6, 2024

(30) Foreign Application Priority Data
Jun. 6, 2023 (CN) 202310659611.6

(51) Int. Cl. G06F 30/23; G06F 119/02

(52) U.S. Cl. CPC G06F 30/23; G06F 2119/02

(57) ABSTRACT

A performance evaluation apparatus includes: a storage portion that stores multiple analysis data, an evaluation portion, and an acquisition portion. The analysis data includes: first analysis data obtained by analyzing first input data, acquired by obtaining a load in first direction acting on the vibration proof rubber over a preset time, using finite element method; and second analysis data obtained by analyzing second input data, acquired by obtaining a load in second direction acting on the vibration proof rubber over the preset time, using the finite element method. The evaluation portion generates: a first matrix in which multiple first coordinate data acquired by performing coordinate transformation on the first analysis data are used as a coordinate plane; and a second matrix in which multiple second coordinate data acquired by performing coordinate transformation on the second analysis data are used as a coordinate plane. The evaluation portion sets a priority order.

### CROSS-REFERENCE TO RELATED APPLICATION

This application claims the priority benefit of China application serial no. 202310659611.6, filed on Jun. 6, 2023. The entirety of the above-mentioned patent application is hereby incorporated by reference herein and made a part of this specification.

### TECHNICAL FIELD

The disclosure relates to performance evaluation apparatus for a vibration proof rubber.

### DESCRIPTION OF RELATED ART

In the development of vibration proof rubbers in vehicles, the endurance test takes a long time (e.g., three months), and it also takes time to master the endurance performance. In addition, if the required target performance is not achieved, the development must be reworked, such as performing the endurance test again. In addition, the endurance test for the vibration proof rubbers with different design information (e.g., shape, size, material) requires multiple data acquisition, and the endurance test takes a long time.

For example, Japanese Patent Application Laid-Open No. JP2009020066A (Patent Document 1) discloses a life estimating method for rubber product, which uses the finite element method (FEM) to analyze the rubber products to obtain output data and to create a matrix based on the output data, so as to determine the life of rubber products. The finite element method refers to subdividing the regions of the target product into multiple meshes and analyzing the change amount in each mesh to predict the overall behavior. Although Patent Document 1 is for the rubber product of a specific shape, it does not describe how the designer uses the life estimating results, for example, selecting for better vibration proof rubber based on the life estimating results. The method disclosed in Patent Document 1 has a problem that it is necessary to further shorten the time required to determine the life of rubber products.

For example, in the initial concept stage of development, it is necessary to select a vibration proof rubber that meets the standard (specification) from vibration proof rubbers that differ in shape and/or material. However, since the standard include various items, it is necessary to select a vibration proof rubber that satisfies these different items. Thus, the help of knowledge and experience is required in the selection. Accordingly, how to select the vibration proof rubber to meet different items is one of the issues that needs to be improved.

### SUMMARY

The disclosure relates to a performance evaluation apparatus for a vibration resistant rubber, which is capable of easily selecting the design of the vibration proof rubber.

According to the embodiment of the disclosure, the performance evaluation apparatus for the vibration proof rubber includes: a storage portion, storing multiple analysis data of the vibration proof rubber acquired by changing design information of the vibration proof rubber; an evaluation portion, generating a matrix based on the multiple analysis data; and an acquisition portion, acquiring a threshold value corresponding to at least one evaluation item in the analysis data. The analysis data includes: first analysis data obtained by analyzing first input data using finite element method, in which the first input data is acquired by obtaining a load in a first direction acting on the vibration proof rubber over a preset time; and second analysis data obtained by analyzing second input data using the finite element method, in which the second input data is acquired by obtaining a load in a second direction acting on the vibration proof rubber over the preset time. The evaluation portion generates: a first matrix in which multiple first coordinate data acquired by performing coordinate transformation on the first analysis data are used as a coordinate plane; and a second matrix in which multiple second coordinate data acquired by performing coordinate transformation on the second analysis data are used as a coordinate plane. The evaluation portion sets a priority order by performing weighting processing on the first coordinate data and the second coordinate data based on the threshold value acquired through the acquisition portion.

Based on the above, this disclosure uses the first analysis data and the second analysis data acquired by changing the design information (e.g., shape, size, material) of the vibration proof rubber to create the first matrix and the second matrix, and perform weighting processing respectively. The first analysis data is related to the endurance in the first direction (e.g., radial direction) of the vibration proof rubber. The second analysis data is related to the endurance in the second direction (e.g., torsion direction) of the vibration proof rubber. Accordingly, even using multiple analysis data acquired by changing the design information of the vibration proof rubber, the user (designer) may easily select a design of the vibration proof rubber that is close to the required threshold value (standard), thereby reducing the time required in the initial concept stage of development. In addition, even if the user (designer) has little knowledge or experience in selecting the suitable vibration proof rubber to meet the standard, he/she may easily select the design of the vibration proof rubber.

### BRIEF DESCRIPTION OF THE DRAWINGS

FIG. 1 is a block diagram of the performance evaluation apparatus of the vibration proof rubber according to an embodiment of the disclosure.

FIG. 2 is a block diagram of the performance evaluation apparatus of the vibration proof rubber according to another embodiment of the disclosure.

FIG. 3A is a schematic view of the first matrix according to an embodiment of the disclosure.

FIG. 3B is a schematic view of the second matrix according to an embodiment of the disclosure.

FIG. 4 is a schematic view of the matrix when multiple coordinate points present on the same coordinates on the third virtual straight line according to an embodiment of the disclosure.

FIG. 5A is a schematic view for illustrating the radial direction according to an embodiment of the disclosure.

FIG. 5B is a schematic view for illustrating the torsion direction according to an embodiment of the disclosure.

FIG. 5C and FIG. 5D are schematic views for illustrating the axial direction according to an embodiment of the disclosure.

FIG. 5E is a schematic view of the prying direction according to an embodiment of the disclosure.

### DESCRIPTION OF THE EMBODIMENTS

Reference will now be made in detail to the exemplary embodiments of the disclosure, and examples of the exemplary embodiments are illustrated in the accompanying drawings. Whenever possible, the same reference numerals are used in the drawings and the description to indicate the same or similar parts.

In the performance evaluation apparatus for the vibration proof rubber in the embodiment of the disclosure, the acquisition portion acquires a first threshold value and a second threshold value based on a first endurance target and acquires a third threshold value and a fourth threshold value based on a second endurance target. The evaluation portion: sets a first virtual straight line on each vertical axis of the first matrix and the second matrix based on the first threshold value and the third threshold value, respectively; sets a second virtual straight line on each horizontal axis of the first matrix and the second matrix based on the second threshold value and the fourth threshold value, respectively; sets a third virtual straight line extending at a designated angle using an intersection point of the first virtual straight line and the second virtual straight line as an original point; and performs a first weighting processing, in a specific region partitioned by the first virtual straight line and the second virtual straight line, through performing a relative evaluation on multiple coordinate points located within the specific region, and the multiple coordinate points are intersection points of the third virtual straight line and the multiple first coordinate data and the multiple second coordinate data.

In the performance evaluation apparatus for the vibration proof rubber in the embodiment of the disclosure, the evaluation portion: performs (a), (b), and (c) below when multiple coordinate points are present on same coordinates on the third virtual straight line: (a) performing a second weighting processing through performing relative evaluation on multiple coordinate points located in the first matrix and the second matrix, which are intersection points of the first virtual straight line and the multiple first coordinate data and the multiple second coordinate data; (b) performing a third weighting processing through performing relative evaluation on multiple coordinate points located in the first matrix and the second matrix, which are intersection points of the second virtual straight line and the multiple first coordinate data and the multiple second coordinate data; and (c) calculating a total score by adding results of the first weighting processing, results of the second weighting processing, and results of the third weighting processing in the first matrix and the second matrix, respectively.

In the performance evaluation apparatus for the vibration proof rubber in the embodiment of the disclosure, the acquisition portion further acquires weighting-related information set by an user, and the evaluation portion changes a weighting factor multiplied by at least one of the results of the first weighting processing, the results of the second weighting processing, and the results of the third weighting processing based on the weighting-related information.

In the performance evaluation apparatus for the vibration proof rubber in the embodiment of the disclosure, the evaluation portion adds a total score of the first matrix and a total score of the second matrix for each piece of design information.

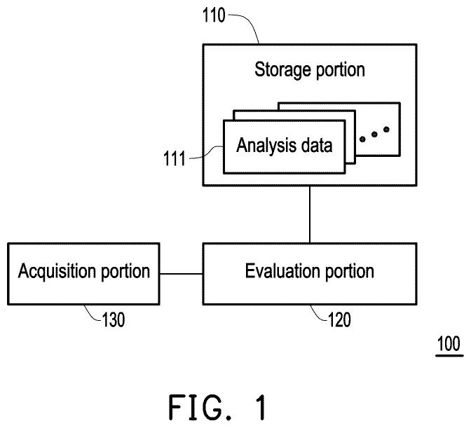

FIG. 1 is a block diagram of the performance evaluation apparatus of the vibration proof rubber according to an embodiment of the disclosure. Referring to FIG. 1, the performance evaluation apparatus 100 includes a storage portion 110, an evaluation portion 120, and an acquisition portion 130. In an embodiment, the performance evaluation apparatus 100 may be electronic devices with computing functions such as a smartphone, a tablet, and a personal computer.

The storage portion 110 stores multiple analysis data 111 of the vibration proof rubber acquired by changing design information of the vibration proof rubber. In addition, the storage portion 110 further includes one or more code segments, which is executed by the evaluation portion 120 after being installed. The storage portion 110 is, for example, fixed or mobile random access memory (RAM), read-only memory (ROM), cache, hard disk, or other similar devices or combination thereof.

The endurance of the part is calculated based on various combinations of shapes, material fatigue characteristics, etc., of multiple design templates through performing fatigue endurance simulation using the finite element method in advance, and the “analysis data 111” is stored in the database of the storage portion 110. This is known as the performance catalog. Thus, the designer may design in real time without having to calculate each time. In addition to the material data under constant temperature, the fatigue characteristics and heat aging characteristics under high temperature are stored, and the endurance performance data combined with the shape thereof (design data) are further stored and used for designing. Whether in terms of shape or material, the endurance of tensile strength may be provided in the same way as providing the dimension data of the part.

The evaluation portion 120 generates a matrix based on the multiple analysis data 111. The evaluation portion 120 may be implemented using physical hardware such as a central processing unit (CPU), a physical processing unit (PPU), a microprocessor, a digital signal processor (DSP), and an electronic control unit (ECU).

The acquisition portion 130 is configured to acquire a threshold value corresponding to at least one evaluation item in the analysis data 111. For example, the acquisition portion 130 is a human machine interface (HMI), and the user may input at least one threshold value using the acquisition portion 130 and provide the threshold value to the evaluation portion 120.

For example, the evaluation portion 120 performs coordinate transformation on the multiple analysis data 111 to acquire multiple coordinate data and a matrix in which these coordinate data are used as the coordinate plane. Next, the evaluation portion 120 performs weighting processing on the multiple coordinate data based on the threshold value acquired by the acquisition portion 130, thereby determining the priority orders of the multiple analysis data 111.

Further details are provided in the embodiment below. In the embodiment below, weighting is performed on the shape of the vibration proof rubber and two fatigue-related matrixes are shown.

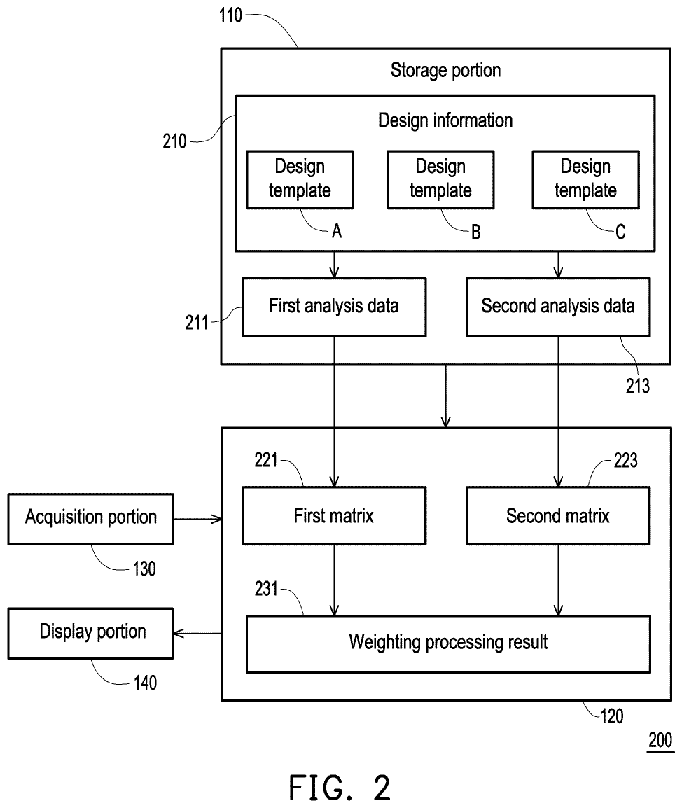

FIG. 2 is a block diagram of the performance evaluation apparatus of the vibration proof rubber according to another embodiment of the disclosure. Referring to FIG. 2, the performance evaluation apparatus 200 includes the storage portion 110, the evaluation portion 120, the acquisition portion 130, and a display portion 140. The performance evaluation apparatus 200 in this embodiment is an application example of the performance evaluation apparatus 100. The performance evaluation apparatus 200 of this embodiment further includes the display portion 140. The display portion 140 is, for example, a display such as a liquid crystal display (LCD) or a plasma display.

Multiple design information 210 of the vibration proof rubber are stored in the storage portion 110. The design information 210 includes, for example, the shape, size, and material of the vibration proof rubber. Different kinds of design templates may be acquired by changing the design information 210. Here, design templates A, B, and C are used for illustration, but the design is not limited thereto. For example, the spring rate of the design template A is 10000 N/mm, the spring rate of the design template B is 8000 N/mm, and the spring rate of the design template C is 12000 N/mm.

In this embodiment, acquiring the first analysis data 211 and the second analysis data 213 using the loads acting in the first direction and the second direction on the vibration proof rubber, respectively, is described. The first direction is, for example, a radial direction, the second direction is, for example, a torsion direction, but it is not limited thereto. The first analysis data 211 and the second analysis data 213 may be acquired, for example, in the following ways: (1) machine learning, automatically calculated based on general bushing finite element method (FEM) analysis data and the design templates A, B, and C; (2) directly calculated by FEM analysis of bushings in the design templates A, B, and C; and (3) experimental data.

Specifically, on vibration proof rubbers based on different design templates, the first input data (including three sets of first input data corresponding to the design templates A, B, and C) are acquired by obtaining the load acting in the first direction over a preset time. Afterwards, the first analysis data 211 (including three sets of first analysis data corresponding to the design templates A, B, and C) is acquired through analyzing the first input data using the FEM. For example, for the vibration proof rubber of the design template A, the first input data of the design template A is acquired by obtaining the load acting in the first direction over the preset time, and then the first input data of the design template A is analyzed using the FEM to acquire the first analysis data 211 of the design template A, and so on for the design templates B and C.

Moreover, on vibration proof rubbers based on different design templates, the second input data (including three sets of second input data corresponding to the design templates A, B, and C) are acquired by obtaining the load acting in the second direction over a preset time. Afterwards, the finite element method (FEM) is used to analyze the second input data to acquire the second analysis data 213. That is, the second analysis data 213 (including three sets of second analysis data corresponding to the design templates A, B, and C) includes data corresponding to multiple design templates. The second analysis data 213 includes multiple sets of analysis data corresponding to multiple design samples.

The evaluation portion 120 generates a first matrix 221 and a second matrix 223 based on the first analysis data 211 and the second analysis data 213, respectively. The evaluation portion 120 acquires a weighting processing result 231 through performing weighting processing on the first matrix 221 and the second matrix 223, respectively, according to the threshold values acquired by the acquisition portion 130. Furthermore, the evaluation portion 120 further displays the weighting processing result 231 on the display portion 140.

In addition, in other embodiments, the direction of the load acting on the vibration proof rubber is not limited to the radial direction and the torsion direction. For example, the endurance for other axial loads or other loads may be a third matrix or a fourth matrix, and each matrix may be further weighted.

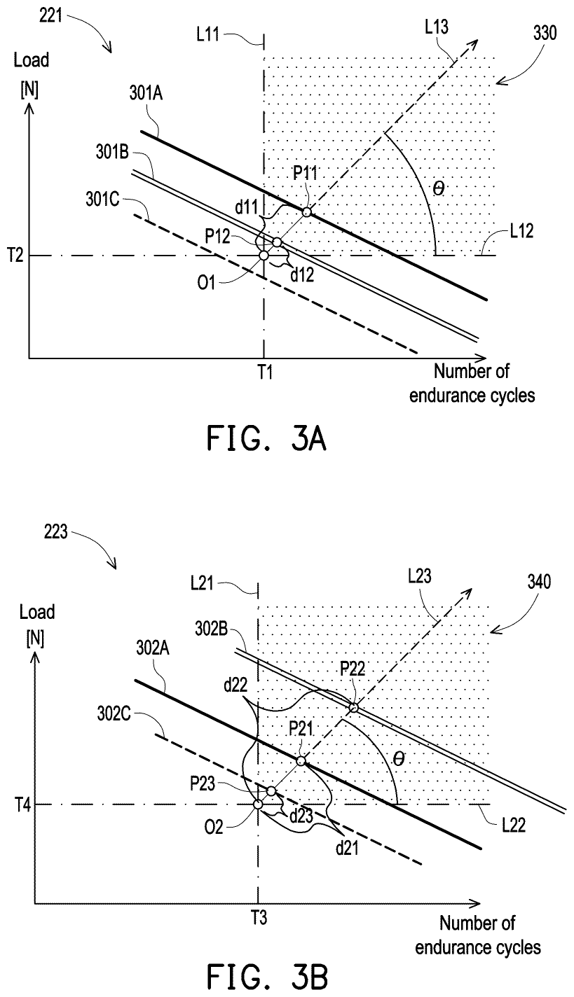

FIG. 3A and FIG. 3B below further describe how the weighting processing on the first matrix 221 and the second matrix 223 are performed. FIG. 3A is a schematic view of the first matrix according to an embodiment of the disclosure. FIG. 3B is a schematic view of the second matrix according to an embodiment of the disclosure. In this embodiment, the design templates A, B, and C are used for description, but it is not limited thereto. For example, designs D, E, F, etc., may also be added.

In FIG. 3A, the evaluation portion 120 performs coordinate transformation on the first analysis data 211 of the design templates A, B, and C to acquire the first coordinate data 301A, 301B, and 301C respectively corresponding to the design templates A, B, and C and generate the first matrix 221 in which the first coordinate data 301A, 301B, and 301C are used as the coordinate plane. In FIG. 3B, the evaluation portion 120 performs coordinate transformation on the second analysis data 213 of the design templates A, B, and C to acquire multiple second coordinate data 302A, 302B, and 302C respectively corresponding to the design templates A, B, and C and generate the second matrix 223 in which the second coordinate data 302A, 302B, and 302C are used as the coordinate plane.

The acquisition portion 130 acquires a first threshold value T1 and a second threshold value T2 based on a first endurance target (e.g., with radial endurance as the evaluation item) and a third threshold value T3 and a fourth threshold value T4 based on a second endurance target (e.g., with torsional endurance as the evaluation item). The evaluation portion 120 partitions specific regions 330 and 340 in the first matrix 221 and the second matrix 223, respectively, according to the first threshold value T1 to the fourth threshold value T4, so as to perform a relative evaluation for multiple coordinate points in the specific regions 330 and 340 to perform the first weighting processing.

That is, the evaluation portion 120 performs the first weighting processing for the first matrix 221 acquired based on the load acting in the first direction (i.e., the radial direction) to acquire the radial endurance scores of the design templates A, B, and C. Furthermore, the evaluation portion 120 performs the first weighting processing for the second matrix 223 acquired based on the load acting in the second direction (i.e., the torsion direction) to acquire the torsional endurance scores of the design templates A, B, and C. The specific regions 330 and 340 may be displayed on the display portion 140 so that the user may intuitively judge whether it is good or not, thereby improving usability.

Specifically, as shown in FIG. 3A, the evaluation portion 120 provides a first virtual straight line L11 on the vertical axis of the first matrix 221 based on the first threshold value T1 and a second virtual straight line L12 on the horizontal axis of the first matrix 221 based on the second threshold value T2. The first virtual straight line L11 and the second virtual straight line L12 partition a specific region 330, and the evaluation portion 120 performs the first weighting processing for the first endurance target (radial endurance) through performing the relative evaluation on multiple coordinate points located within the specific region 330.

In the embodiment shown in FIG. 3A, the evaluation portion 120 provides a third virtual straight line L13 extending within the specific region 330 at a designated angle θ using an intersection point of the first virtual straight line L11 and the second virtual straight line L12 as an original point O1. Here, the designated angle θ is, for example, 45 degrees, but not limited thereto, and the designated angle θ may also be set to be within a range. The relative evaluation is performed on multiple coordinate points (e.g., coordinate point P11 and coordinate point P12) located on the third virtual straight line L13 within the specific region 330 as the first weighting processing. In this embodiment, only the coordinate point P11 and the coordinate point P12 where the first coordinate data 301A and 301B intersect the third virtual straight line L13 are located within the specific region 330.

Here, when the evaluation target is related to endurance, the higher the endurance, the higher the evaluation. Therefore, the evaluation portion 120 selects the one having the farthest distance from the original point O1, that is, the coordinate point P11, among the coordinate point P11 and the coordinate point P12. Next, the evaluation portion 120 sets the score of the original point O1 to 0 and the score of the coordinate point P11 to 100. The score of the coordinate point P11 represents the score corresponding to the situation of the first endurance target of the design template A. Then, for other coordinate points on the third virtual straight line L13, that is, the coordinate point P12, the score of the designed coordinate point P12 is calculated according to the ratio of the distance d11 between the coordinate point P11 and the original point O1 to the distance d12 between the coordinate point P12 and the original point O1. The score of the coordinate point P12 represents the score corresponding to the situation of the first endurance target of the design template B. For example, the score of the coordinate point P12=100×(d12/d1l).

In addition, since the first coordinate data 301C corresponding to the design template C does not intersects the third virtual straight line L13 within the specific region 330, the score of the design template C corresponding to the situation of the first endurance target is set to 0. Accordingly, through the above calculation method, the scores of the design templates A, B, and C corresponding to the situation of the first endurance target (radial endurance) are 100, 10, and 0, respectively.

On the other hand, as shown in FIG. 3B, the evaluation portion 120 provides a first virtual straight line L21 on the vertical axis of the second matrix 223 based on the third threshold value T3 and a second virtual straight line L22 on the horizontal axis of the second matrix 223 based on the fourth threshold value T4. The first virtual straight line L21 and the second virtual straight line L22 partition a specific region 340, and the evaluation portion 120 performs the first weighting processing for the second endurance target (torsional endurance) through performing the relative evaluation on multiple coordinate points located within the specific region 340.

Similar to the first weighting processing for the first endurance target mentioned above, the evaluation portion 120 performs the relative evaluation on multiple coordinate points (e.g., coordinate point P21, coordinate point P22, and coordinate point P23) located on the third virtual straight line L13 within the specific region 340. The evaluation portion 120 sets the score of the original point O2 (the intersection point of the first virtual straight line L21 and the second virtual straight line L22) to 0 and the score of the coordinate point P22 having the farthest distance from the original point O2 to 100. The score of the coordinate point P21 is calculated according to the ratio of the distance d21 between the coordinate point P21 and the original point O2 to the distance d22 between the coordinate point P22 and the original point O2. The score of the coordinate point P23 is calculated according to the ratio of the distance d23 between the coordinate point P23 and the original point O2 to the distance d22 between the coordinate point P22 and the original point O2. For example, the score of the coordinate point P21 100×(d21/d22); the score of the coordinate point P23=100×(d23/d22). Accordingly, the scores of the design templates A, B, and C corresponding to the torsional endurance are 50, 100, and 20, respectively.

In an embodiment, the evaluation portion 120 sets the priority orders for the design templates A, B, and C based on the scores in terms of two endurance targets (e.g., radial endurance and torsional endurance) of the design templates A, B, and C. For example, the priority orders are set based on the total score acquired by adding the scores corresponding to the two endurance targets. For example, the evaluation portion 120 adds the total score of the first matrix 221 (related to the radial endurance) and the total score of the second matrix 223 (related to the torsional endurance) of the design templates A, B, and C, respectively, and the priority orders are set based on the summed total score. Table 1 lists the weighting processing result of an embodiment. In the example shown in Table 1, the priority orders of the design templates A, B, and C are: design template A→design template B→design template C, from the highest to the lowest, respectively.

TABLE 1

| | Score | | |
|---|---|---|---|
| Design | Radial | Torsional | Total |
| template | endurance | endurance | score |
| A | 100 | 50 | 150 |
| B | 10 | 100 | 110 |
| C | 0 | 20 | 20 |

In an embodiment, the first matrix 221 (radial endurance) and the second matrix 223 (torsional endurance) are displayed simultaneously on the display portion 140 for the user to intuitively see and thus easily grasp that the design template A has excellent radial endurance but poor torsional endurance; the design template B has poor radial endurance but excellent torsional endurance; and the design template C is less favorable in both radial endurance and torsional endurance. Furthermore, the scores in Table 1 may also be presented by images rather than numerical values.

In other embodiments, the evaluation portion 120 may further calculate the scores of the static spring value and the dynamic spring value. The evaluation portion 120 sets the priority orders for the design templates A, B, and C based on the scores corresponding to both the static spring value and the dynamic spring value by adding the scores in terms of the two endurance targets (e.g., radial endurance target and torsional endurance) of the design templates A, B, and C.

For example, the priority orders are set based on the total score acquired by adding the scores corresponding to the two endurance targets and the scores corresponding to both the static spring value and the dynamic spring value. Table 2 lists the weighting processing result of an embodiment. In the example shown in Table 2, the priority orders of the design templates A, B, and C are: design template A→design template B→design template C, from the highest to the lowest, respectively.

TABLE 2

| | Score | | | | |
|---|---|---|---|---|---|
| Design | Radial | Torsional | Static | Dynamic | Total |
| template | endurance | endurance | spring value | spring value | score |
| A | 100 | 50 | 80 | 75 | 305 |
| B | 10 | 100 | 100 | 100 | 310 |
| C | 0 | 20 | 66 | 55 | 141 |

In an embodiment, the score corresponding to the static spring value is calculated as follows. For the static spring values (i.e., the spring rates) of the design templates A, B, and C of 10000 N/mm, 8000 N/mm, and 12000 N/mm, respectively, in the case where the target value is set at less than or equal to 13000 N/mm, among the spring rates that meet the target value, the lower the spring rate, the higher the score. Thus, the score of the design template B, which corresponds to the smallest value of the above spring rates, is set to 100, and the scores of the design templates A and C are calculated based on the ratio of the other spring rates.

The score of the design template B=100;
the score of the design template A=(spring rate of the design template B÷ spring rate of the design template A)×score of the design template B=8000+10000×100=80; and
the score of the design template C=(spring rate of the design template B÷ spring rate of the design template C)×score of the design template B=8000+12000×100=66.667.

In Table 2, the integer “66” represents the score corresponding to the static spring value of the design template C. Accordingly, the corresponding scores of the design templates A, B, and C in terms of the static spring value are 80, 100, and 66, respectively.

The scores of the dynamic spring values of the design templates A, B, and C may also be calculated through the above calculation method. For example, the scores in terms of the dynamic spring values of the design templates A, B, and C are 75, 100, and 55, respectively.

The design gives priority to noise and vibration and ride comfort, so the softer the spring, the better. Accordingly, a calculation formula is adopted in which the lower the spring rate, the higher the score.

On the other hand, if mobility and stability are given priority, the spring should be harder. Thus, contrary to prioritizing ride comfort, a calculation formula is adopted in which the higher the spring rate, the higher the score. For example, assuming that the static spring values of the design templates A, B, and C are 10000 N/mm, 8000 N/mm, and 12000 N/mm, respectively, when the target value is set to be greater than or equal to 7000 N/mm, then among the spring rates that meet the target value, the higher the spring rate, the higher the score. Thus, given that the score of the design template C corresponding to the maximum value of the spring rates is 100, the scores of the design templates A and B are calculated based on the ratio between other spring rates.

In addition, in order to balance noise and vibration, ride comfort, and handing stability, the target value of the spring rate is established within a specified range. That is, the median of the specified range becomes the maximum score, and the closer the spring rate is to the minimum or maximum value in the specified range, the lower the score. For example, in the case that the target value is set to be greater than or equal to 7000 N/mm and less than or equal to 13000 N/mm, then among the spring rates that meet the target value, the median (10000 N/mm) scores the highest. Thus, given that the score of the design template A corresponding to the median of the spring rates is set to 100, the scores of the design templates B and C are calculated based on the ratio between other spring rates.

The target values of the dynamic spring value and the score calculation method are similar to the calculation of the corresponding scores of the static spring values.

In other embodiments, the user may input the weighting-related information using the acquisition portion 130 to further adjust the weighting processing result. For example, the weighting-related information is set as follows: weighting factor of radial endurance=1; weighting factor of torsional endurance=1.3; weighting factor of static spring value=1; and weighting factor of dynamic spring value=1. By adjusting the example shown in Table 2 based on the weighting-related information, the results shown in Table 3 below may be acquired.

TABLE 3

| | Score after multiplying by a weighting factor | | | | |
|---|---|---|---|---|---|
| Design | Radial | Torsional | Static | Dynamic | Total |
| template | endurance | endurance | spring value | spring value | score |
| A | 100 | 65 | 80 | 75 | 320 |
| B | 10 | 130 | 100 | 100 | 340 |
| C | 0 | 26 | 66 | 55 | 147 |

Since the weighted results may be determined based on the performance valued by the user, cycle fatigue performance evaluation that reflects the user's tendencies and trends may be provided and the time required in the initial concept stage of development may be reduced. For example, in other embodiments, when the weighting factor of torsional endurance is adjusted to 1.5 and the other weighting factors are still 1, the total scores of the design templates A, B, and C change to 330, 360, and 151. Thus, in the case that the user values the torsional endurance, the priority orders are adjusted as: design template B→design template A→design template C, from the highest to the lowest.

In another embodiment, when multiple coordinate points present on the same coordinates on the third virtual straight line, the following (a), (b), and (c) are performed: (a) performing a second weighting processing through performing relative evaluation on multiple coordinate points located in the first matrix and the second matrix on the first virtual straight line; (b) performing a third weighting processing through performing relative evaluation on multiple coordinate points located in the first matrix and the second matrix on the second virtual straight line; and (c) calculating a total score by adding results of the first weighting processing, results of the second weighting processing, and results of the third weighting processing in the first matrix and the second matrix, respectively.

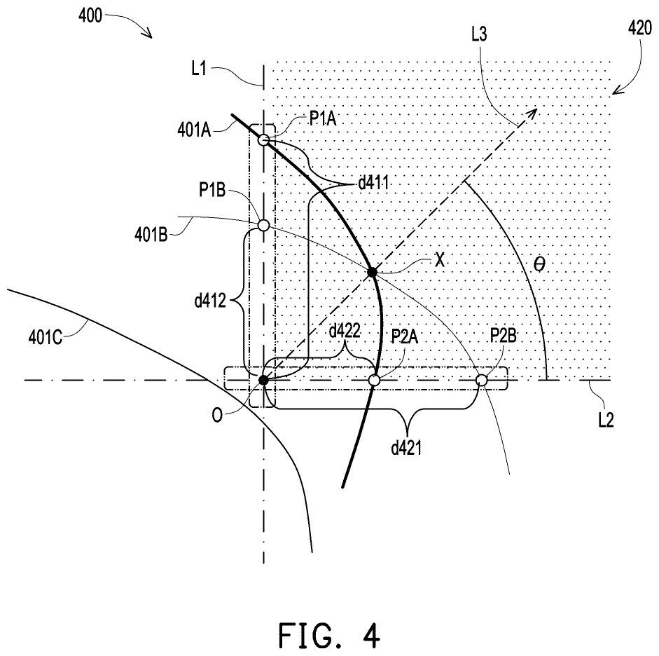

FIG. 4 is a schematic view of the matrix when multiple coordinate points present on the same coordinates on the third virtual straight line according to an embodiment of the disclosure. The matrix 400 shown in FIG. 4 may be the first matrix generated based on the load in the first direction or the second matrix generated based on the load in the second direction.

Referring to FIG. 4, the matrix 400 includes the first coordinate data 401A, 401B, and 401C corresponding to the design templates A, B, and C, the first virtual straight line L1 and the second virtual straight line L2 partition a specific region 420, and the third virtual straight line L3 extends within the specific region 420 at the designated angle θ. Furthermore, the intersection point of the first virtual straight line L1 and the second virtual straight line L2 is the original point O.

As shown in FIG. 4, the coordinate points of both the first coordinate data 401A and 401B present on the same coordinates on the third virtual straight line L3, that is, the first coordinate data 401A and 401B and the third virtual straight line L3 intersect at the coordinate point X. In this case, the relative evaluation is performed on the multiple coordinate points on the third virtual straight line L3, the first virtual straight line L1, and the second virtual straight line L2 to perform the first weighting processing, the second weighting processing, and the third weighting processing, respectively.

For performing the first weighting processing on the coordinate points located in the specific region 420 on the third virtual straight line L3, the score of the farthest point from the original point O is set to 100, and the score of other points are set in proportion to the distance from the original point O. Detailed calculations are described in FIG. 3A and FIG. 3B above. In the example of FIG. 4, since the first coordinate data 401A and 401B and the third virtual straight line L3 intersect at the coordinate point X, and the coordinate point X is the farthest point from the original point O, the scores corresponding to the design templates A and B are both set to 100. Since the first coordinate data 401C corresponding to the design template C does not intersect with the third virtual straight line L3 in the specific region 420, the score corresponding to the design template C is set to 0.

For performing the second weighting processing on the coordinate points located in the specific region 420 on the first virtual straight line L1, the score of the farthest point from the original point O is set to 100, and the score of other points are set in proportion to the distance from the original point O. That is, the score of coordinate point P1A=100; the score of coordinate point P1B=the score of coordinate point P1A×(distance d412 between the coordinate point P1B and the original point O/distance d411 between the coordinate point P1A and the original point O).

For performing the third weighting processing on the coordinate points located in the specific region 420 on the second virtual straight line L2, the score of the farthest point from the original point O is set to 100, and the score of other points are set in proportion to the distance from the original point O. That is, the score of coordinate point P2B=100; the score of coordinate point P2A=the score of coordinate point P2B×(distance d422 between the coordinate point P2A and the original point O/distance d421 between the coordinate point P2B and the original point O).

Table 4 below lists the result of the first weighting processing, the result of the second weighting processing, and the result of the third weighting processing shown in FIG. 4. Moreover, the result of the first weighting processing, the result of the second weighting processing, and the result of the third weighting processing are added for the design templates A, B, and C, respectively, to acquire the total scores corresponding to the design templates A, B, and C.

TABLE 4

| | Score | | | |
|---|---|---|---|---|
| | Result of first | Result of second | Result of third | |
| Design | weighting | weighting | weighting | Total |
| template | processing | processing | processing | score |
| A | 100 | 100 | 50 | 250 |
| B | 100 | 80 | 100 | 280 |
| C | 0 | 0 | 0 | 0 |

Through the above method, even if there are multiple design templates with the same score in the result of the first weighting processing, the second weighting processing and the third weighting processing may still be performed by the relative evaluation on the multiple coordinate points located on the first virtual straight line and the second virtual straight line. Based on these weighting processing results, a rubber design close to the required threshold value (standard) is designed, thereby reducing the time required in the initial concept stage of development.

In another embodiment, the weighting-related information set by the user may be further acquired using the acquisition portion 130. The evaluation portion 120 changes a weighting factor multiplied by at least one of the results of the first weighting processing, the results of the second weighting processing, and the results of the third weighting processing based on the weighting-related information.

The radial endurance and the torsional endurance have been described above, but the disclosure is not limited thereto, and the axial endurance and the prying direction endurance may also be evaluated. In addition, combined tests may also be performed on these endurance evaluation items to evaluate the test results. For example, after prying, torsion is applied to evaluate the endurance performance by repeatedly applying loads in the radial direction. Thus, each endurance evaluation item may be processed individually and a matrix (performance catalog information) of the endurance evaluation items may be created by combining multiple endurance evaluation items.

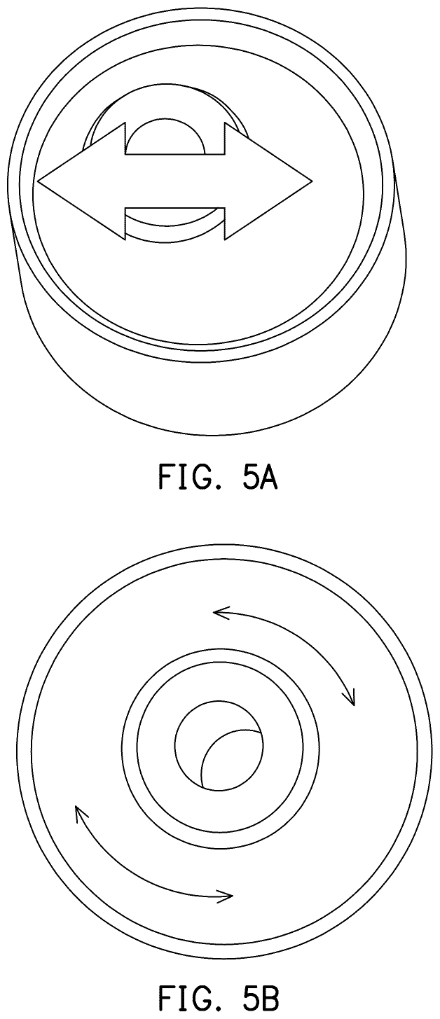

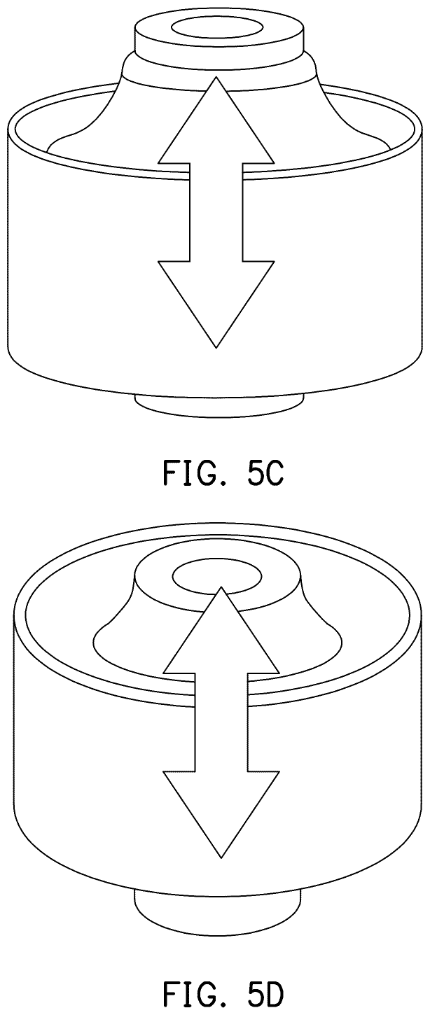

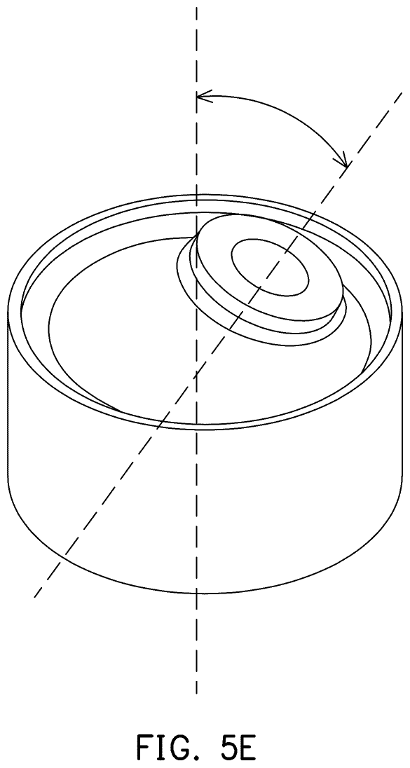

FIG. 5A is a schematic view for illustrating the radial direction according to an embodiment of the disclosure. FIG. 5B is a schematic view for illustrating the torsion direction according to an embodiment of the disclosure. FIG. 5C and FIG. 5D are schematic views for illustrating the axial direction according to an embodiment of the disclosure. FIG. 5E is a schematic view of the prying direction according to an embodiment of the disclosure. As shown by the arrow in FIG. 5A, the radial direction is a linear direction along the diameter or radius of the bearing surface. As shown by the arrow in FIG. 5B, the torsion direction may be a clockwise direction or a counterclockwise direction. As shown by the arrows in FIG. 5C and FIG. 5D, the axial direction is the direction of the center axis of rotation of the cylinder, i.e., the same direction as the central axis, and the axial direction is perpendicular to the radial direction. As shown by the arrow in FIG. 5E, the prying direction is the direction orthogonal to the centerline.

Finally, it should be noted that the above embodiments are only used to illustrate the technical solutions of the disclosure and are not intended to limit it. Although the disclosure has been described in detail with reference to the above embodiments, persons of ordinary skill in the art should understand that they may still modify the technical solutions described in the above embodiments, or replace some or all of the technical features therein with equivalents, and that such modifications or replacements of corresponding technical solutions do not substantially deviate from the scope of the technical solutions of the embodiments of the disclosure.

What is claimed is:

1. A performance evaluation apparatus for a vibration proof rubber, comprising:
a storage portion, storing a plurality of analysis data of the vibration proof rubber acquired by changing design information of the vibration proof rubber;
an evaluation portion, generating a matrix based on the plurality of analysis data; and
an acquisition portion, acquiring a threshold value corresponding to at least one evaluation item in the analysis data;
wherein the analysis data comprises:
first analysis data obtained by analyzing first input data using finite element method, wherein the first input data is acquired by obtaining a load in a first direction acting on the vibration proof rubber over a preset time; and
second analysis data obtained by analyzing second input data using the finite element method, wherein the second input data is acquired by obtaining a load in a second direction acting on the vibration proof rubber over the preset time,
wherein the evaluation portion generates:
a first matrix in which a plurality of first coordinate data acquired by performing coordinate transformation on the first analysis data are used as a coordinate plane; and
a second matrix in which a plurality of second coordinate data acquired by performing coordinate transformation on the second analysis data are used as a coordinate plane, and
the evaluation portion sets a priority order by performing weighting processing on the first coordinate data and the second coordinate data based on the threshold value acquired through the acquisition portion.

2. The performance evaluation apparatus for the vibration proof rubber according to claim 1, wherein
the acquisition portion acquires a first threshold value and a second threshold value based on a first endurance target and acquires a third threshold value and a fourth threshold value based on a second endurance target, and
the evaluation portion:
sets a first virtual straight line on each vertical axis of the first matrix and the second matrix based on the first threshold value and the third threshold value, respectively;
sets a second virtual straight line on each horizontal axis of the first matrix and the second matrix based on the second threshold value and the fourth threshold value, respectively;
sets a third virtual straight line extending at a designated angle using an intersection point of the first virtual straight line and the second virtual straight line as an original point; and
performs a first weighting processing, in a specific region partitioned by the first virtual straight line and the second virtual straight line, through performing a relative evaluation on a plurality of coordinate points located within the specific region, and the plurality of coordinate points are intersection points of the third virtual straight line and the plurality of first coordinate data and the plurality of second coordinate data.

3. The performance evaluation apparatus for the vibration proof rubber according to claim 2, wherein the evaluation portion:
performs (a), (b), and (c) below when a plurality of coordinate points are present on same coordinates on the third virtual straight line:
(a) performing a second weighting processing through performing relative evaluation on a plurality of coordinate points located in the first matrix and the second matrix, which are intersection points of the first virtual straight line and the plurality of first coordinate data and the plurality of second coordinate data;
(b) performing a third weighting processing through performing relative evaluation on a plurality of coordinate points located in the first matrix and the second matrix, which are intersection points of the second virtual straight line and the plurality of first coordinate data and the plurality of second coordinate data; and
(c) calculating a total score by adding results of the first weighting processing, results of the second weighting processing, and results of the third weighting processing in the first matrix and the second matrix, respectively.

4. The performance evaluation apparatus for the vibration proof rubber according to claim 3, wherein
the acquisition portion further acquires weighting-related information set by a user, and
the evaluation portion changes a weighting factor multiplied by at least one of the results of the first weighting processing, the results of the second weighting processing, and the results of the third weighting processing based on the weighting-related information.

5. The performance evaluation apparatus for the vibration proof rubber according to claim 3, wherein
the evaluation portion adds a total score of the first matrix and a total score of the second matrix for each piece of design information.

6. The performance evaluation apparatus for the vibration proof rubber according to claim 4, wherein
the evaluation portion adds a total score of the first matrix and a total score of the second matrix for each piece of design information.
